(19) United States
(12) Patent Application Publication
(10) Pub. No.: US 2009/0253998 A1
(43) Pub. Date: Oct. 8, 2009

(54) SKIN BIOPSY WITH SUTURING PRIOR TO RESECTION

(75) Inventor: RAYMOND H. CHEN, San Marino, CA (US)

Correspondence Address: MCDERMOTT WILL & EMERY LLP;38th Floor, 2049 Century Park East, Los Angeles, CA, 90067, US

(73) Assignee: Convergent Medical Solutions, Inc.

(21) Appl. No.: 12/062,408

(22) Filed: Apr. 3, 2008

(51) Int. Cl. A61B 10/02

(52) U.S. Cl. 600/565; 600/564

(57) ABSTRACT

An automated skin biopsy apparatus for taking a biopsy of a lesion on skin may include a suture applicator configured to controllably suture skin surrounding the lesion before the lesion is resected from the skin, a lesion resector configured to controllably resect the lesion from the skin, a user-operated control, and a controller. The controller may be configured, in response to actuation of the user-operated control by a user, to automatically cause the suture applicator to suture skin surrounding the lesion and the lesion resector to resect the lesion from the skin while the sutures from the suture applicator are in place. Related processes are disclosed.

### BACKGROUND

1. Field

This disclosure relates to skin biopsies and to robotics.

2. Description of Related Art

Skin lesions can be problematic, such as when they are cancerous or contain pre-cancerous cells. A biopsy can be performed to help diagnose whether the skin lesion should be of concern.

Unfortunately, many family physicians often lack the skill to perform a biopsy. Family physicians may also be hesitant to refer a patient to a specialist out of concern for costs. Sometimes, the patient is part of an insurance plan that even penalizes the physician for such a referral. As a consequence, problematic skin lesions are sometimes not promptly diagnosed and treated.

### SUMMARY

An automated skin biopsy apparatus for taking a biopsy of a lesion on skin may include a suture applicator configured to controllably suture skin surrounding the lesion before the lesion is resected from the skin, a lesion resector configured to controllably resect the lesion from the skin, a user-operated control, and a controller. The controller may be configured, in response to actuation of the user-operated control by a user, to automatically cause the suture applicator to suture skin surrounding the lesion and the lesion resector to resect the lesion from the skin while the sutures from the suture applicator are in place.

The automated skin biopsy apparatus may include a lesion stabilizer configure to controllably stabilize the lesion while on the skin. The controller may be configured to automatically cause the lesion stabilizer to stabilize the lesion while on the skin and before the suture applicator sutures the skin in response to the actuation of the user-operated control by the user.

The lesion stabilizer may be configured to controllably hold the lesion after it has been resected from the skin by the lesion resector. The controller may be configured to automatically cause the lesion stabilizer to hold the lesion after it has been resected from the skin by the lesion resector in response to the actuation of the user-operated control by the user.

The lesion stabilizer may be configured to controllably release the lesion after it has been resected from the skin by the lesion resector. The controller may be configured to cause the lesion stabilizer to release the lesion after it has been resected from the skin by the lesion resector.

The lesion stabilizer may include a suction tube configured to apply suction to the lesion and a suction pump configured to controllably deliver suction to the suction tube. The controller may be configured to automatically cause the suction pump to deliver suction to the suction tube in response to the actuation of the user-operated control by the user.

The suction tube may have a suction pathway and include a screen mounted within the suction pathway. The controller may be configured to automatically cause the suction pump to stop delivering suction to the suction tube after the lesion has been resected from the skin.

The lesion stabilizer may include a gripper configured to controllably grip the lesion. The controller may be configured to cause the gripper to grip the lesion in response to the actuation of the user-operated control by the user.

The lesion stabilizer may be configured to controllably pull the lesion away from the skin. The controller may be configured to automatically cause the lesion stabilizer to pull the lesion away from the skin in response to the actuation of the user-operated control by the user.

The automated skin biopsy apparatus may include an anesthesia applicator configured to controllably apply anesthesia in the area of the lesion. The controller may be configured to automatically cause the anesthesia applicator to apply anesthesia in the area of the lesion before the suture applicator sutures the skin in response to the actuation of the user-operated control by the user.

The anesthesia applicator may include a plurality of needles.

The automated skin biopsy apparatus may include a camera configured to take a photograph of the lesion before it is resected from the skin by the lesion resector.

The automated skin biopsy apparatus may include a printer configured to print a label for a specimen container that is configured to hold the resected lesion. The controller may be configured to cause the printer to print a label with information that identifies a patient associated with the lesion.

The lesion resector may include at least one cutting blade.

The suture applicator and the lesion resector may both by part of a single tool.

An skin biopsy process for taking a biopsy of a lesion on skin may include suturing skin surrounding the lesion and resecting the lesion from the skin after the suturing and while the sutures are in place.

The skin biopsy process may include initiating an automated process and the suturing and the resecting may be part of the automated process.

The automated process may include stabilizing the lesion while on the skin before the suturing.

The stabilizing may include applying suction to the lesion.

The automated process may include pulling the lesion away from the skin before the resecting.

The automated process may include holding the lesion in a holding area after the resecting.

The stabilizing may include griping the lesion.

The automated process may include pulling the lesion away from the skin before the resecting.

The automated process may include applying anesthesia in the area of the lesion after the stabilizing and before the resecting.

The applying anesthesia may be performed by injecting anesthesia through a plurality of needles simultaneously.

These, as well as other components, steps, features, objects, benefits, and advantages, will now become clear from a review of the following detailed description of illustrative embodiments, the accompanying drawings, and the claims.

### BRIEF DESCRIPTION OF DRAWINGS

The drawings disclose illustrative embodiments. They do not set forth all embodiments. Other embodiments may be used in addition or instead. Details that may be apparent or unnecessary may be omitted to save space or for more effective illustration. Conversely, some embodiments may be practiced without all of the details that are disclosed. When the same numeral appears in different drawings, it is intended to refer to the same or like components or steps.

FIG. 1 illustrates an automated skin biopsy apparatus.

FIG. 2 is an enlarged view of the suction tube illustrated in FIG. 1.

FIG. 3 illustrates a gripper that may be used in lieu of the suction tube illustrated in FIG. 1.

FIG. 4 illustrates an enlarged view of the injection needle illustrated in FIG. 1.

FIG. 5 illustrates a multiple needle injector that may be used in lieu of the single needle injector illustrated in FIGS. 1 and 4.

FIG. 6 illustrates an injector having a local supply of anesthesia that may be used in lieu of the injector illustrated in FIG. 1.

FIG. 7 illustrates an enlarged view of the suture applicator illustrated in FIG. 1.

FIG. 8 illustrates a suture applicator that may be used in lieu of the suture applicator illustrated in FIG. 1.

FIG. 9 illustrates an enlarged view of the cutting tool illustrated in FIG. 1.

FIG. 10 illustrates a combined suture and cutting tool that may be used in lieu of the separate suture applicator and cutting tool illustrated in FIG. 1.

FIG. 11 is a flow diagram of steps that may be performed prior to activation of an automated skin biopsy process.

FIGS. 12(a)-(i) illustrate an automated skin biopsy process, along with a manual step that may precede it.

FIG. 13 is a flow diagram of an automated skin biopsy process.

### DETAILED DESCRIPTION OF ILLUSTRATIVE EMBODIMENTS

Illustrative embodiments are now discussed. Other embodiments may be used in addition or instead. Details that may be apparent or unnecessary may be omitted to save space or for a more effective presentation. Conversely, some embodiments may be practiced without all of the details that are disclosed.

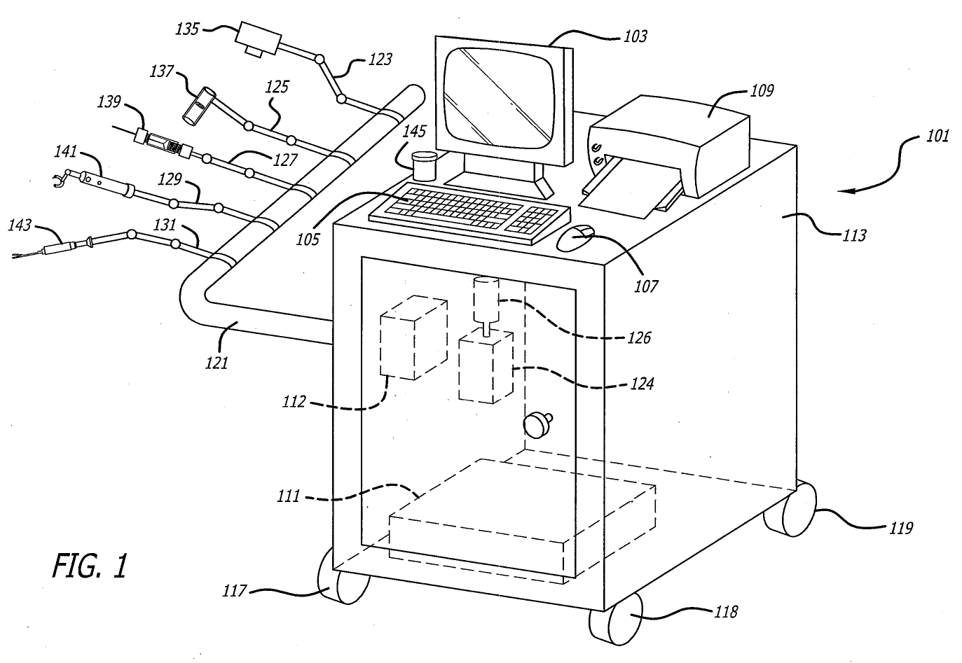

FIG. 1 illustrates an automated skin biopsy apparatus. As shown in FIG. 1, a control console 101 may include a user interface, such as a display 103, a keyboard 105, a mouse 107, and a printer 109. The control counsel may include a controller 111, a station housing 113 mounted on wheels 117 and 119, a main arm 121 connected to branch arms 123, 125, 127, 129 and 131, a camera 135 connected to the branch arm 123, a lesion stabilizer such as a suction tube 137 connected to the branch arm 125 and an associated suction pump 112 within the station housing 113, an anesthesia applicator such as an injection needle 139 connected to the branch arm 127 and an associated anesthesia pump 124 and anesthesia supply 126, a suture applicator 141 connected to the branch arm 129, and a lesion resector such as a cutting tool 143 connected to the branch arm 131.

A specimen container 145 may rest on the control console 101. The specimen container 145 may be configured to hold a biopsy of a skin lesion. It may be of any type, shape or size.

The camera 135, the suction tube 137, the injection needle 139, the suture applicator 141, and the cutting tool 143 are tools whose locations in three dimensional space may be controlled by electro-mechanical actuators that may be located in or near the joints of the branch arms to which they are attached, as well as by electro-mechanical actuators that may be located in or near the joint between each of the branch arms and the main arm 121. These electro-mechanical actuators may convert electrical energy into movement of the joints in or near which they are located, such a bending, straightening and/or rotation of the joints of the branch arms and rotation of the joint between each branch arm and the main arm. The electro-mechanical actuators may be of any type and may include one or more solenoids, motors, ball screws, gears, pulleys, belts and/or other mechanisms to effectuate these movements.

These actuators, in turn, may each be actuated and controlled by one or more electrical signals from the controller 111. The combined effect of actuating these actuators may be to allow the controller 111 to move each of the tools to which each branch arm is connected to any desired position and orientation in three-dimensional space that is within the reach of the branch arms. Other forms of linkages and associated electro-mechanical actuators may be used in addition or instead. For example, all of the tools may be attached to a single rotating turret that, in turn, is attached to a single, jointed arm. The rotational position of the turret and the joints in the single arm may be controlled by actuators so as to similarly position any of the tools anywhere in three dimensional space that is within reach of the apparatus. The actuators, in turn, may be driven and controlled by the controller 111. Pneumatic or other types of controllers and actuators may be used in addition or instead.

The various tools and/or the branch arms to which they are attached may also include associated position sensors so that the controller 111 may know the exact position in three-dimensional space at which each tool resides or is moved. This information may be used by the controller 111 to enable the controller to position one tool with respect to a position to which another tool has been manually moved by a user. This information may also be used by the controller 111 in a feedback loop so as to enable the controller 111 to position each tool precisely. Any type of position sensors may be used, including sensors that are directly linked to the tools and/or image recognition technology.

Other forms of robotic apparatuses and control systems may be used in addition or instead. Additional tools or a lesser number of tools may also be used.

The camera 135 may be a digital camera capable of taking high definition, close up photographs of a skin lesion or other part of a body. The camera 135 may be configured to deliver an electronic signal representative of that image to the controller 111 thorough an appropriate communication system.

The branch arm 123 to which the camera 135 is affixed may be configured to allow the branch arm to be manually positioned by the physician. The branch arm 123 may not have actuators or associated position sensors. In other embodiments, the position of the camera 135 may be controlled by the controller 111 in which case the branch arm 123 may include both actuators and position sensors.

The controller 111 may be located within the control console 101 and/or elsewhere. The controller 111 may be configured to drive the actuators so as to cause each of the tools to move to the positions that are described below, as well as to cause each of the tools and other devices that are described herein to perform each of the functions that are described below.

To accomplish this, the controller 111 may include appropriate hardware and software. For example, the controller 111 may include a general purpose computer, such as a PC, and/or a dedicated computer. The controller 111 may include one or more processing systems, permanent memory, such as one or more hard drives and/or ROMS, and temporary memory, such as one or more RAMs. One or more computer programs may run in the controller 111 and may be stored in one or more of the permanent memories, along with patient information, files containing photographic images taken by the camera 135, and other information. The controller 111 may be connected to external sources of data, such as to one or more patient databases and patient record systems, through one or more networks or other types of communication system.

The automated skin biopsy apparatus that is illustrated in FIG. 1 may be located in a room containing a table on which a patient may lie, a chair on which a patient may sit, and/or a chair on which a physician may sit. The physician's chair may have wheels to enable the physician to move between the patient and the user interface components on the control console 101.

The lesion stabilizer may be configured to controllably stabilize a lesion on the skin of a patient while other procedures take place. One example of such a lesion stabilizer is the suction tube 137 and suction pump 112 illustrated in FIG. 1.

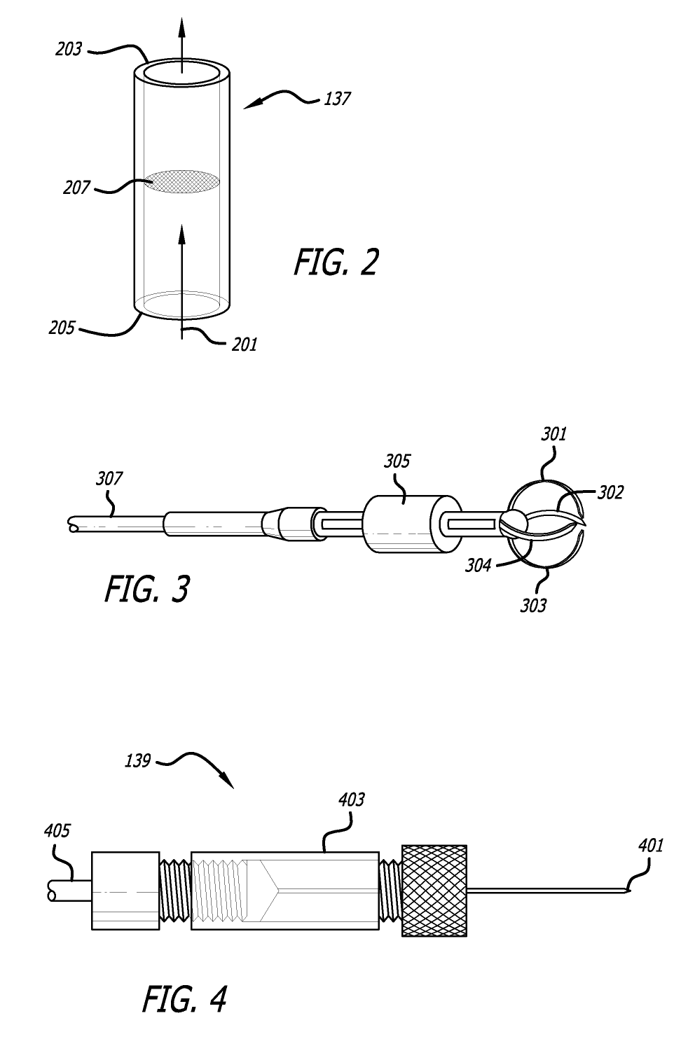

FIG. 2 is an enlarged view of the suction tube 137 that is illustrated in FIG. 1. As illustrated in FIG. 2, the suction tube 137 may have a suction pathway 201 through which suction may be applied from a connection end 203 of the suction tube 137 to a patient end 205 of the suction tube 137. Within this pathway, a biopsy trap, such as a screen 207, may be positioned so as to trap a biopsy when it travels through the suction pathway 201 under the force of suction. This may enable the biopsy to be trapped before it is sucked into the control console 101, as will be described below in more detail. It may also provide a convenient means for the biopsy to be later released into the specimen container 145, as will also be described below.

In a different embodiment, there may be no biopsy trapping device within the suction tube 137. Instead, the biopsy may be allowed to travel through the branch arm 125, through the main arm 121, and into the control console 101, wherein it may be trapped for either manual or automatic delivery into a specimen container.

The wall of the suction tube 137 may be made of a transparent material, such as glass or clear plastic, so as to allow a skin lesion to be viewed through the wall of the suction tube 137 while it is being positioned on top of the skin lesion, as will be described below.

The branch arm 125 to which the suction tube 137 may be affixed, as well as the main arm 121, may be configured to provide a substantially air-tight pathway for suction to be applied by the suction pump 112 to the patient end 205 of the suction tube 137. Alternatively, the suction from the suction pump 112 may be applied to the suction tube 137 through a different pathway.

The suction tube 137 may be configured so as to releasably connect to the end of the branch arm 125, thus allowing the suction tube 137 to be readily attached to and detached from branch arm 125. The releasably connection may maintain a substantially air-tight connection when connected to the suction tube 137.

The suction tube 137 may be cylindrical and of any size. Various other sizes and shapes of suction tubes may be provided. For example, suction tubes of different cross-sectional diameters and shapes may be provided. This may enable the physician to select a particular size or shape which is best suited to taking a biopsy of a lesion of a particular size and type, as will be explained more fully below. Although the patient end 205 of the suction tube 137 is illustrated in FIG. 2 as being planer, it may instead be curved so as to better create a suction seal between the suction tube 137 and a skin surface that is sharply curved, such as a skin surface over a finger.

The suction tube 137 may be configured in a way to enable information about its size and shape to be automatically communicated to the controller 111. For example, the suction tube 137 may include an internal data chip that includes this information and that is linked to the controller 111 through the branch arm 125 and the main arm 121. The suction tube 137 may instead be configured with surface indentations or other irregularities that mate with corresponding surface sensors in the branch arm 125 to similarly provide size and configuration information to the controller 111 automatically.

Although being shown as within the station housing 113, the suction pump 112 may be elsewhere, such as in the handle portion of the branch arm 125 to which the suction tube 137 attaches.

A suction tube and an associated suction pump are not the only type of lesion stabilizer that may be used. For example, a mechanical gripping device may be used in addition or instead.

FIG. 3 illustrates a gripper that may be used in lieu of the suction tube illustrated in FIG. 1. As shown in FIG. 3, the gripper may include a set of opposing jaws 301 and 303 that can be controllably opened and closed by an electro-mechanical actuator 305 that may be linked through linkage 307 to the branch arm 125. In this embodiment, the controller 111 may be configured to position the gripper and to control the position of the opposing jaws 301 and 303 in accordance with the processes described below.

The anesthesia applicator may be of any type. For example, it may be the injection needle 139 illustrated in FIG. 1.

FIG. 4 illustrates an enlarged view of the injection needle illustrated in FIG. 1. As illustrated in FIG. 4, the injection needle 139 may include a sharp hollow needle 401 attached to a cylinder 403 which may communicate through a tube 405 through the branch arm 127 and the main arm 121 with the anesthesia pump 124. The anesthesia pump, in turn, may be configured to controllable pump anesthesia through the injection needle 139 from the anesthesia supply 126 under the control of the controller 111. Connecting tubing may be provided within the station housing 113, the main arm 121, and the branch arm 127 to facilitate the transfer of the anesthesia from the anesthesia supply 126 through the injection needle 139. The controller may be configured to activate and control the pumping action of the anesthesia pump 124.

The injection needle in FIG. 4 is illustrated as having only a single needle 401. In other configurations, anesthesia may be injected simultaneously through a set of needles.

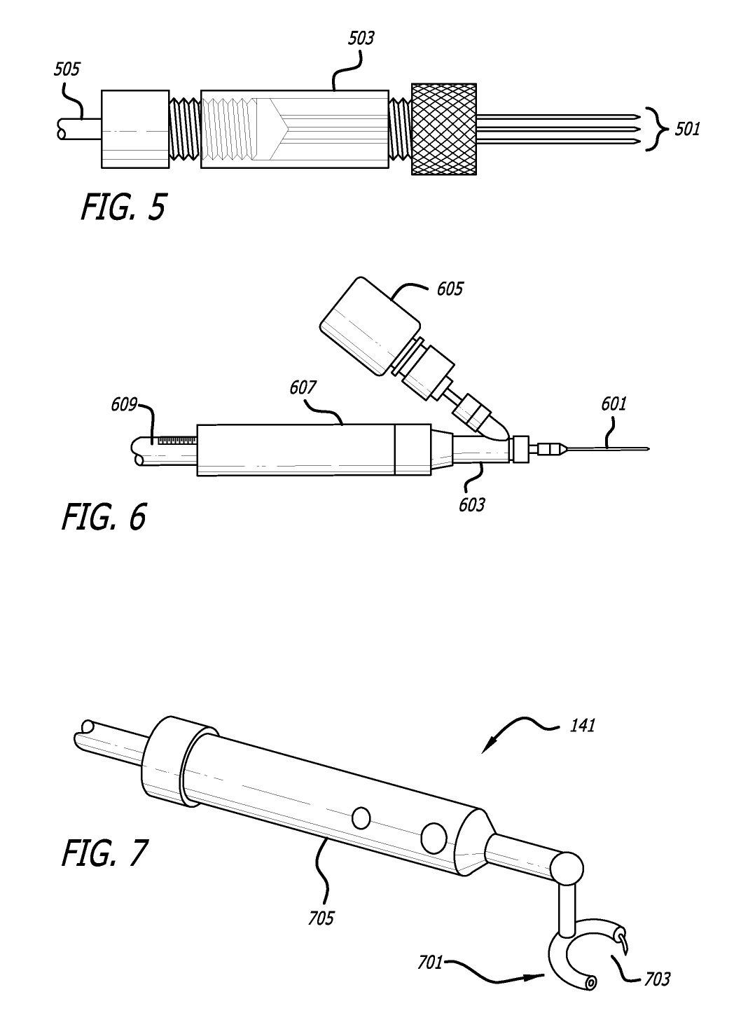

FIG. 5 illustrates a multiple needle injector that may be used in lieu of the single needle injector illustrated in FIGS. 1 and 4. As illustrated in FIG. 5, the multiple needle injector may include multiple hollow needles 501 attached to a cylinder 503 which may be fed by an anesthesia feed tube 505.

FIG. 6 illustrates an injector having a local supply of anesthesia that may be used in lieu of the needle injector illustrated in FIGS. 1 and 4. As illustrated in FIG. 6, a hollow needle 601 may be connected to a tube 603 and to a local supply of anesthesia 605. A control tube 607 may be provided and may be connected via linkage 609 to the branch arm 125. In this configuration, an electrical, pneumatic or other type of signal may be supplied through the linkage 609 to cause the local supply of anesthesia 605 to be injected through the hollow needle 601 in a controllable amount under the control of the controller 111.

FIG. 7 illustrates an enlarged view of the suture applicator illustrated in FIG. 1. As shown in FIG. 7, the suture applicator 141 may have a suture applicator head 701 having a jaw-like access port 703 that may be attached to a storage compartment 705 that may be connected to the branch arm 129. A supply of suture material may be stored in the storage compartment 705. Under the control of the controller 111, the suture applicator 141 may utilize one or more internal actuators to cause a curved needle to travel circuitously through the jaw-like access port 703, repeatedly pulling suture material through tissue that is placed within the access port 703.

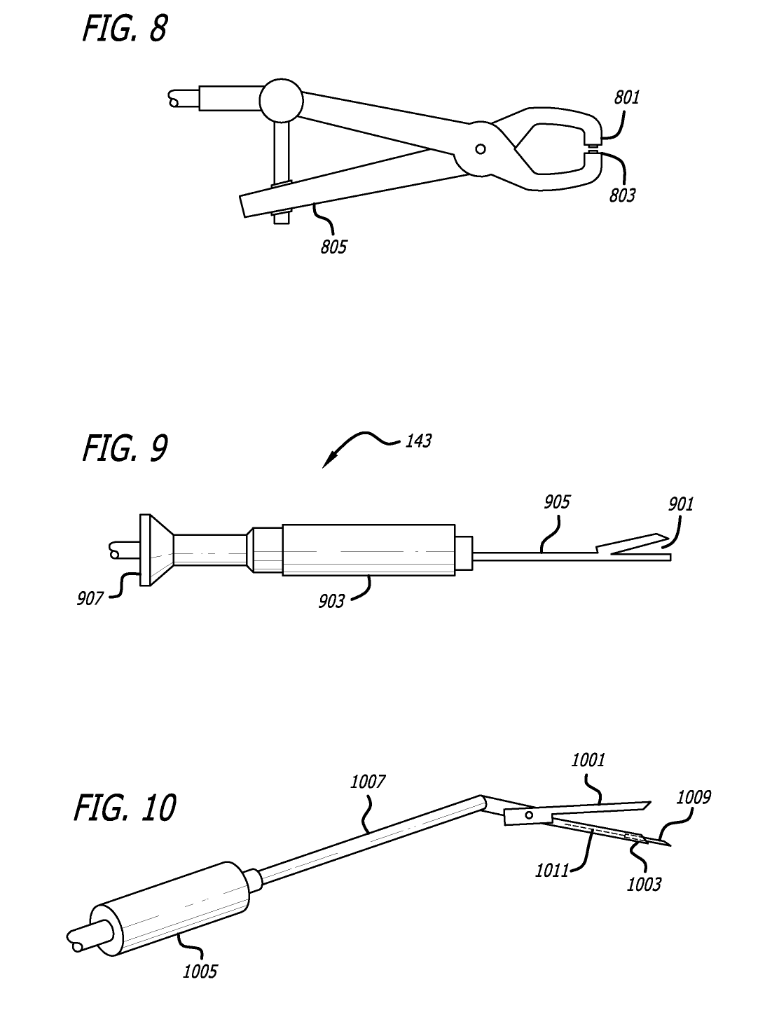

FIG. 8 illustrates a suture applicator that may be used in lieu of the suture applicator illustrated in FIG. 1. The suture applicator illustrated in FIG. 8 may include a set of jaw members 801 and 803 and a jaw actuator 805 that may controllable cause the jaw members 801 and 803 to open and close. The jaw actuator 805 may also cause one or more staples in one of the jaw members to pierce tissue placed within the jaw and to be folded over at the other end by the other jaw member in a stapling-like action. In this embodiment, staple-like sutures may be applied under the control of the controller 111 through the application of appropriate control signals.

The lesion resector may be configured to controllably resect the lesion or a portion of it from the skin. Any type of lesion resector may be used, such as the cutting tool 143 illustrated in FIG. 1.

FIG. 9 illustrates an enlarged view of the cutting tool illustrated in FIG. 1. As illustrated in FIG. 9, the cutting tool 143 may include a cutting channel 901 that is configured to cut tissue that is inserted within the cutting channel 901 under the control of an actuator 903 that communicates with the cutting channel 901 through linkage 905. The cutting channel 901 may include one or more cutting blades. The cutting tool 143 may include linkage 907 that connects to the branch arm 131. Under the control of the controller 111, the cutting tool 143 may cut tissue.

FIG. 10 illustrates a combined suture and cutting tool that may be used in lieu of the separate suture applicator and cutting tool illustrated in FIG. 1. As illustrated in FIG. 10, the combined tool may include opposing jaws 1001 and 1003 which may cooperate to both suture tissue placed within the jaw with a suture needle 1009 and cutting blade 1011, together with an actuator 1005 which may control the cutting and suturing of the opposing jaws 1001 and 1003 through a linkage 1007. The combined suture and cutting tool may be connected to one of the branch arms in FIG. 1, such as the branch arm 129 or 131. Under the control of the controller 111, the combined suture and cutting tool that is illustrated in FIG. 10 may both suture and cut tissue.

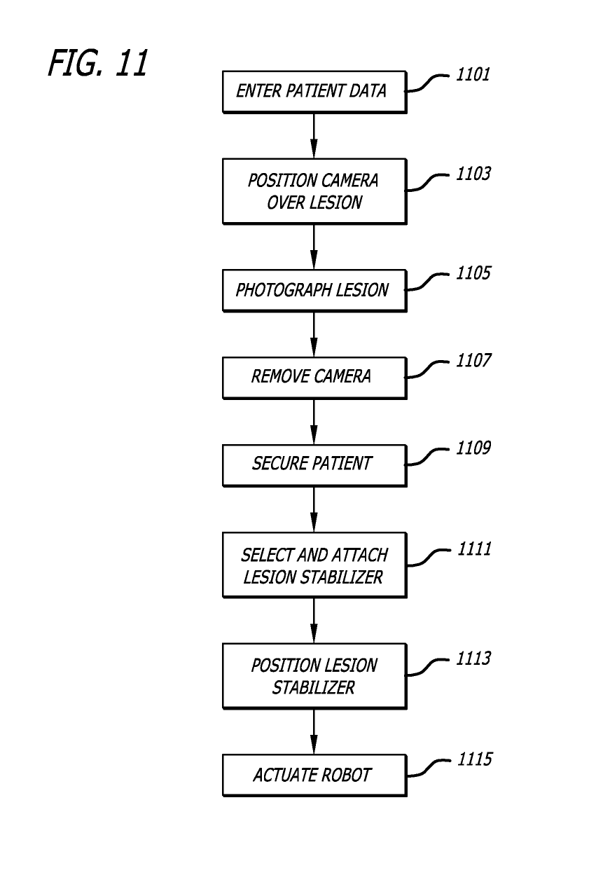

FIG. 11 is a flow diagram of steps that may be performed prior to actuation of an automated skin biopsy process. The automated skin biopsy apparatus illustrated in FIG. 1 may be used to implement the process shown in FIG. 11. Other types of automated skin biopsies biopsy apparatus may also be used to implement this process. The automated skin biopsy apparatus illustrated in FIG. 1 may also be used to implement other processes, not described by FIG. 11.

Data concerning a patient may be entered, as illustrated by an Enter Patient Data step 1101 in FIG. 11. During this step, information about the patient may be entered through the user interface, such as by using the keyboard 105 and the mouse 107 while watching the display 103. Any type of information may be entered, such as a name of a patient, a patient number, a patient's address, a phone number and/or an e-mail address. Medical information may also be entered about the patient, such as observations relating to a skin lesion, a diagnoses, a recommended treatment, etc. The controller 111 may be linked to a database of patient information from which further information may be obtained or to which some or all of the entered information may be delivered.

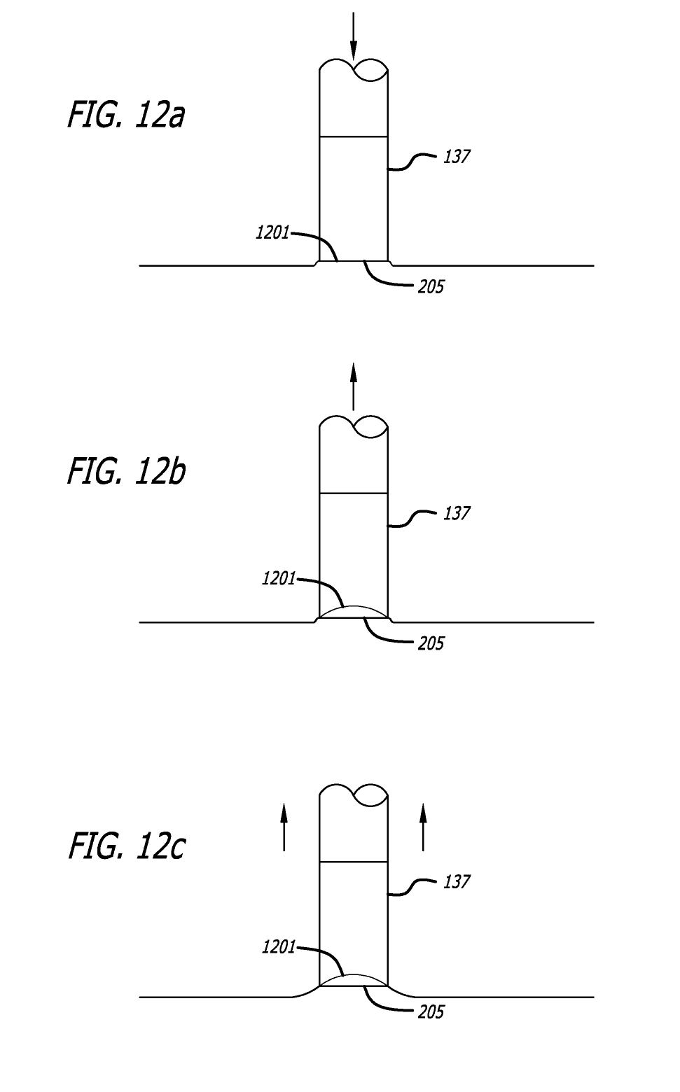

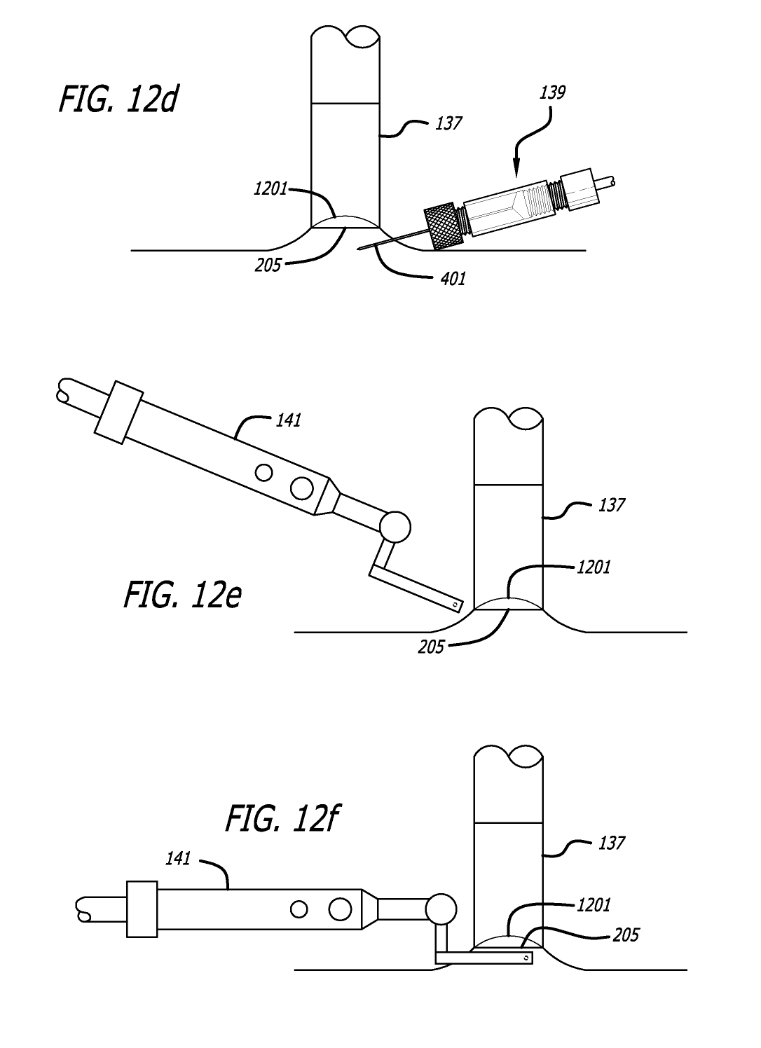

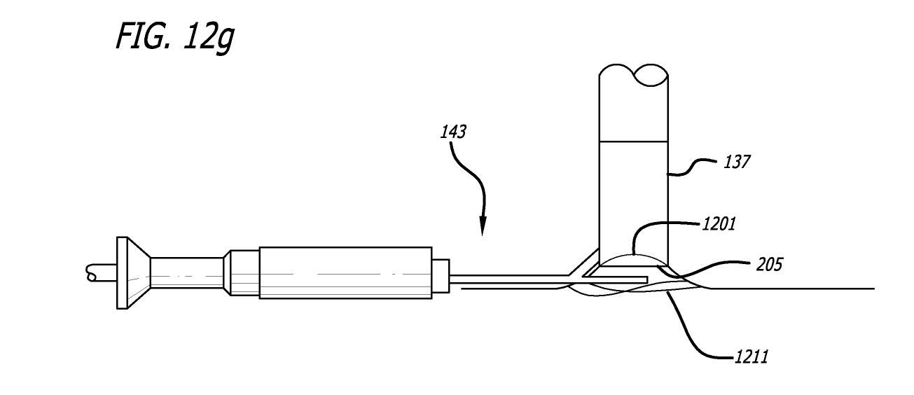

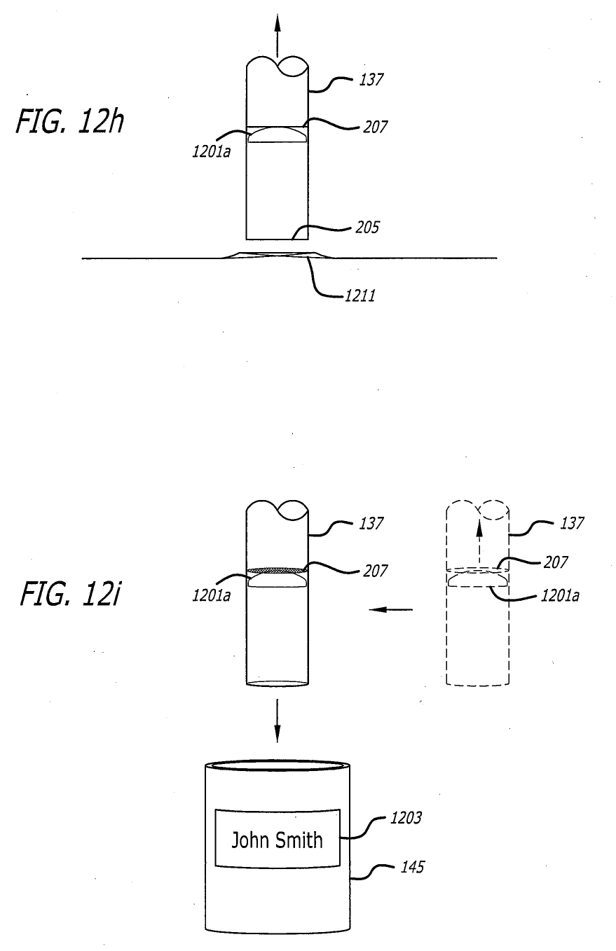

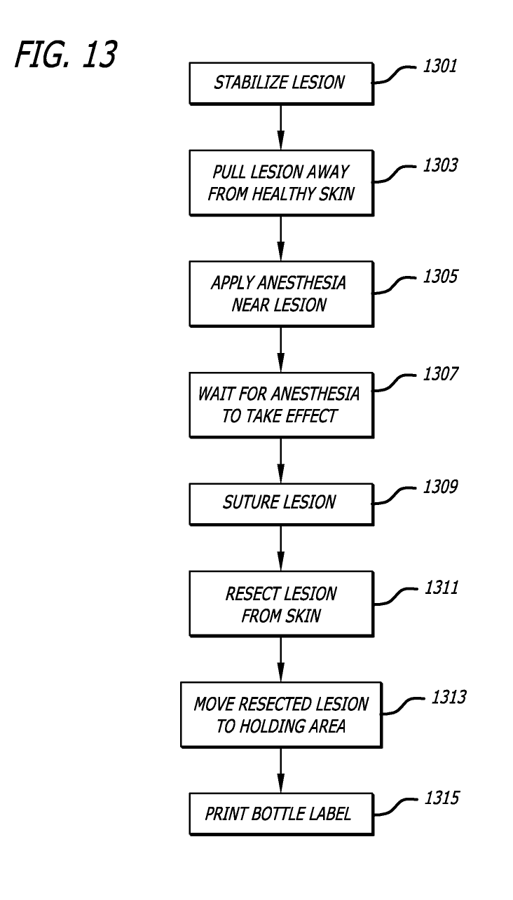

None, some or all of this information may be used by the controller 111 in performing one or more of the automated steps discussed below in connection with FIGS. 12 and 13. For example, some of the entered information may be sued by the controller to determine the size of the biopsy and/or its depth.

The camera 135 may be positioned so as to take a close-up photograph of the skin lesion, as reflected by a Position Camera Over Lesion step 1103. During this step, a physician may manually move the camera to a desired position. Alternatively, the camera 135 may be moved by automated means, either under remote control by the physician, or through the use of image recognition technology. The camera 135 may also be positioned to take photographs of other areas of the body.

An image may also be displayed on the display 103 before the image is captured. This may enable the physician to verify that a desired view has been faithfully framed.

An image of the lesion may be taken, as reflected by a Photograph Lesion step 1105. The camera 135 may be directed to take the image manually or automatically. The image may again or for the first time be displayed on the display 103 to enable the physician to verify that a useful photograph has been taken.

The camera 135 may be moved to other locations, such as to other perspectives of the lesion, and additional photographs may be taken.

The images that are captured may be stored in the controller 111 and/or elsewhere. They may also or instead be printed. The images may be associated with the patient for future reference.

The camera 135 may be moved out-of-the-way, as illustrated by a Remove Camera step 1107.

The area surrounding the lesion to be biopsied may be secured, as reflected by a Secure Patient step 1109, so that movement of the patient does not cause any appreciable corresponding movement of the skin lesion during the automated steps that are described below. For example, if the skin lesion is on the arm of the patient, the arm may be strapped to a stable surface, such as to the top of a table. Similarly, if the lesion is on the back of the patient, stomach may be fastened around the back of the patient, again to prevent movement of the lesion once the automated process begins, as described below.

An appropriate lesion stabilizer may be selected and attached to the branch arm 125, as reflected by a Select and Attach Lesion Stabilizer step 1111. During this step, the physician may select a lesion stabilizer that is appropriate for the lesion to be biopsied. For example, if the lesion is small and round and located on the back, the physician may select a suction tube that is cylindrical, has a small diameter, and has a patient end that is flat. If the lesion is much larger or on a sharply curved surface, on the other hand, a suction tube of a larger size or with a rounded patient end may be selected instead. During some procedures, only a portion of a lesion may be biopsied. During these procedures, a suction tube having a diameter smaller than the diameter of the lesion may be selected. Other types of lesion stabilizers may be used in addition or instead, such as the gripper illustrated in FIG. 3.

Once the lesion stabilizer is selected, it may be attached to the automated skin biopsy apparatus, such as to the branch arm 125 illustrated in FIG. 1. If a lesion stabilizer such as the suction tube 137 is used, a sealant, such as Vaseline, may be placed on the patient end 205 of the suction tube 137.

After attachment, the lesion stabilizer may be positioned, as reflected by a Position Lesion Stabilizer step 1113. During this step, the lesion stabilizer may be placed in a position that is needed prior to initiation of the automated biopsy resection process, as described below.

The exact location at which the lesion stabilizer is positioned may vary, depending upon the type of lesion stabilizer that is used.

FIGS. 12(a)-(i) illustrate an automated skin biopsy process, along with a manual step that may precede it. The process illustrated in FIGS. 12(a)-(j) may be performed by the automated skin biopsy apparatus illustrated in FIG. 1 or by other apparatus. Similarly, the automated skin biopsy apparatus illustrated in FIG. 1 may perform other processes.

As illustrated in FIG. 12(a), the suction tube 137 may then be oriented to be substantially perpendicular to the surface of skin on which a lesion 1201 is present. The patient end 205 of the suction tube 137 may then be rested against the surface of the lesion to be resected. During this positioning, the physician may look through the transparent wall of the suction tube 137 so as to more accurately place it over the area to be resected.

The automated skin biopsy apparatus may then be actuated, as reflected in an Actuate Robot step 1115.

Actuation of the robot may be accomplished by any means. For example, a user may actuate one of the user-operated controls that is part of the user interface to the controller 111, such as by pressing one or more keys on the keyboard 105 and/or sliding the mouse 107 until a mouse pointer moves to a particular location on the display 103 and then actuating a button on the mouse 107. In an alternate embodiment, the display 103 may be a touch display, in which case the physician may merely touch an area on the display 103. In a still further embodiment, a user-operator control may be provided elsewhere on the control console 101, on the main arm 121, on one or more of the branch arms 123, 125, 127, 129 and 131, and/or elsewhere.

Although having now been described and illustrated in a particular sequence, the steps of the process illustrated in FIG. 11 may be performed in a different sequence. The process may also be performed with additional steps or with a smaller number of steps.

FIG. 13 is a flow diagram of an automated skin biopsy process. The process illustrated in FIG. 13 may be performed by the automated skin biopsy apparatus illustrated in FIG. 1 or by other apparatus. Similarly, the automated skin biopsy apparatus illustrated in FIG. 1 may perform other automated processes.

After the user-operated control is actuated, what takes place as illustrated in FIGS. 12(b)-(h) and FIG. 13 may all or partially be automated, i.e., may all take place sequentially without the need for the physician or other person to manually issue any command or take any action.

The first step of the automated process may be to stabilize the lesion, as reflected by a Stabilize Lesion step 1301, i.e, to minimize the ability of the lesion to move during subsequent steps. During this step, the controller 111 may cause the lesion stabilizer to stabilize the lesion.

When the suction tube 137 is being used for the lesion stabilizer, the controller 111 may actuate the suction pump 112, so as to cause suction to be applied to the lesion 1201 through the suction tube 137. This may cause the lesion 1201 to be sucked slightly within the suction tube 137, as illustrated in FIG. 12(b), thus stabilizing it against any appreciable local movement.

Under the control of the controller 111, the electro-mechanical actuators controlling the position of the suction tube 137 may then be actuated so as to cause the suction tube 137 to be pulled slightly away from the surface of the skin, so as to slightly pull the lesion away from the skin, as illustrated in FIG. 12(b), and as indicated by the Pull Lesion Away from Healthy Skin step 1303 in FIG. 13. This is an optional step that may slightly lift the lesion away from the skin so as to make it easier to resect, as discussed below.

The controller 111 may next cause anesthesia to be applied in the area of the lesion, as reflected by an Apply Anesthesia Near Lesion step 1305. During this step, the controller 111 may cause the anesthesia applicator to apply anesthesia near the lesion.

The precise steps that the controller 111 takes to accomplish apply this anesthesia may depend upon the type of anesthesia applicator that is used. If the injection needle 139 is used, for example, the controller 111 may cause the actuators associated with the injection needle 139 to move the injection needle 139 along a pathway that is parallel to the surface of the skin on which the lesion resides, just slightly beneath the patient end 205 of the suction tube 137, until the needle 401 pierces the skin and is properly positioned for an injection, as illustrated in FIG. 12(d). The controller 111 may then activate the anesthesia pump 124 so as to cause a pre-determined amount of anesthesia to be injected through the injection needle into the target site.

In order to move the anesthesia applicator along the correct pathway and to cause it to come to rest at the correct location, information about the position of the patient end 205 of the suction tube 137 after it is rested on the lesion 1201 may be sensed by the position sensors associated with the suction tube 137 and sent to the controller 111. This may provide the controller with a point of reference for use in connection with the positioning of the anesthesia applicator and the performance of other steps of the automated process, such as the positioning of other tools as described below. In other words, knowing the position of the patient end 205 of the suction tube 137 may enable the controller 111 to accurately position the remaining tools for the functions that they perform.

The controller 111 may be pre-programmed with information about the type and size of each tool and the action that each tool must take. This information may also the controller in accurately positioning the tools to enable them to perform the functions for which they are intended.

In some embodiments, anesthesia may be applied at several locations, particularly when a large biopsy is to be taken. To accomplish this, the injection needle 139 may be sequentially moved to these locations and an injection of anesthesia may be made at each. In other embodiments, a multiple needle injector, such as the one illustrated in FIG. 5, may be used instead, requiring only a single positioning operation and pump actuation.

After the anesthesia application is complete, the controller 111 may cause the injection needle 139 to be moved away from the injection site. The controller 111 may then pause for a brief period to allow the anesthesia to take affect.

In some embodiments, the controller may stop the automated process at this point and await to be manually reactivated. In these embodiments, the lesion stabilizer may be removed and the apparatus securing the area of the patient against movement (e.g., limb straps) may also be removed. The patient may then be allowed to roam until the anesthesia has taken affect. Thereafter, the area of the patient from which a biopsy will be taken may be re-secured (e.g., by straps), the lesion may be re-stabilized (e.g., by again placing the suction tube 137 against the lesion and activating the suction pump 112).

In other embodiments, particularly when the anesthesia takes effect quickly, the lesion stabilizer may remain in place while the anesthesia takes effect, e.g., the suction tube 137 may not be moved and the suction may continue to be applied.

In either event, the area under the lesion may next be sutured, as reflected by a suture lesion step 1309. During this step, the controller 111 may cause the area under the lesion to be sutured, thus eliminating the need to suture this area after the biopsy is taken, i.e., at a time when it may be more difficult to locate and stabilize the area that needs to be sutured.

To accomplish this, the controller 111 may direct the suture applicator 141 to apply sutures to the area immediately beneath the patient end 205 of the suction tube 137. The first part of this process may be to command the actuators associated with the branch arm 129 to move the suture applicator 141 to the needed location, as illustrated in FIG. 12(e). Thereafter, the suture applicator 141 may be commanded by the controller 111 to apply sutures 1211, as illustrated in FIG. 12(f).

In order to move the suture applicator 141 to the correct location, the controller 111 may again use the data it received that is indicative of the location of the patient end 205 of the suction pathway 201 as a reference point, as discussed above, together with information relating to the type and size of the suture applicator 141.

The controller 111 may next cause the lesion to be resected from the skin, as reflected by a Resect Lesion From Skin step 1311. During this step, the controller 111 may cause the suture applicator to be moved out of the way and for the lesion resector to be positioned for resecting the lesion, as illustrated in FIG. 12(g). Again, the controller 111 may accomplish this task by making use of the known location of the patient end 205 of the suction tube 137. Once in position, the controller 111 may cause the lesion resector to resect the lesion above the sutures 1211. When the resector is the cutting tool 143, this may cause one or more sharp blades to reciprocate and cut through the lesion.

After the lesion is resected, it may be moved to a holding area, as reflected by a Move Resected Lesion to Holding Area step 1313. During this step, the controller 111 may allow the resected lesion to be sucked into the suction tube 137, as illustrated in FIG. 12(h). The resected lesion 1201(a) may travel this path until it is blocked by the screen 207 in the suction tube 137, as also illustrated in FIG. 12(h).

The controller 111 may cause a label 1203 identifying the patient to be printed on the printer 109, as reflected by a Print Bottle Label step 1315. The label 1203 may be placed on the specimen container 145, as illustrated in FIG. 12(i).

After the lesion is resected, and while it is being held against the screen 207 by suction, the physician may move the suction tube 137 away from the patient and position it over the specimen container 145, as illustrated in FIG. 12(i). Once properly positioned, the physician may signal the controller 111 through an appropriate user interface control to stop the suction pump 112, following which the resected lesion 1201(a) may be released from the suction tube 137 and fall into the specimen container 145. The specimen container 145 may then be sealed. Copies of any photographs that may have been taken may also be printed and may accompany the specimen container 145.

In an alternate embodiment, a cap may instead be placed on the distil end of the suction tube 137, before the suction pump 112 is disabled. The suction pump 112 may then be disabled and the suction tube 137 may then be detached from the branch arm 125 and used as a specimen container itself.

Although now having described the steps of FIG. 13 in a particular sequence, these steps may be performed in a different sequence. Additional steps or a lesser number of steps may also be followed.

The components, steps, features, objects, benefits and advantages that have been discussed are merely illustrative. None of them, nor the discussions relating to them, are intended to limit the scope of protection in any way. Numerous other embodiments are also contemplated, including embodiments that have fewer, additional, and/or different components, steps, features, objects, benefits and advantages. The components and steps may also be arranged and ordered differently.

For example, in lieu of or in addition to injecting anesthesia, skin anesthesia ointment from a tube may be applied to the skin at the site of the lesion and allowed to take effect before the lesion is excised.

The phrase “means for” when used in a claim embraces the corresponding structures and materials that have been described and their equivalents. Similarly, the phrase “step for” when used in a claim embraces the corresponding acts that have been described and their equivalents. The absence of these phrases means that the claim is not limited to any of the corresponding structures, materials, or acts or to their equivalents.

Nothing that has been stated or illustrated is intended to cause a dedication of any component, step, feature, object, benefit, advantage, or equivalent to the public, regardless of whether it is recited in the claims.

In short, the scope of protection is limited solely by the claims that now follow. That scope is intended to be as broad as is reasonably consistent with the language that is used in the claims and to encompass all structural and functional equivalents.

1. An automated skin biopsy apparatus for taking a biopsy of a lesion on skin comprising:
a suture applicator configured to controllably suture skin surrounding the lesion before the lesion is resected from the skin;
a lesion resector configured to controllably resect the lesion from the skin, the lesion resector being configured such that the lesion resector can begin and complete controllably resecting the lesion from the skin after the suture applicator has completed suturing the skin surrounding the lesion;
a user-operated control; and
a controller configured to automatically cause each of the following to occur sequentially in response to and after actuation of the user-operated control by a user without any further command or action being taken by the user:
the suture applicator to suture skin surrounding the lesion; and
after the suture applicator has completed suturing the skin surrounding the lesion the lesion resector to begin and complete resecting the lesion from the skin while the sutures from the suture applicator are in place.

2. The automated skin biopsy apparatus of claim 1 further comprising a lesion stabilizer configured to controllably stabilize the lesion while on and before it is sutured or cut from the skin and wherein the controller is configured to automatically cause the lesion stabilizer to stabilize the lesion while on the skin and before the suture applicator sutures the skin in response to and after the actuation of the user-operated control by the user, without any further command or action being taken by the user.

3. The automated skin biopsy apparatus of claim 2 wherein the lesion stabilizer is configured to controllably hold the lesion after the lesion it-has been resected from the skin by the lesion resector and wherein the controller is configured to automatically cause the lesion stabilizer to hold the lesion after the lesion has been resected from the skin by the lesion resector in response to and after the actuation of the user-operated control by the user without any further command or action being taken by the user.

4. The automated skin biopsy apparatus of claim 3 wherein the lesion stabilizer is configured to controllably release the lesion after the lesion it-has been resected from the skin by the lesion resector and wherein the controller is configured to cause the lesion stabilizer to release the lesion after the lesion it-has been resected from the skin by the lesion resector.

5. The automated skin biopsy apparatus of claim 2 wherein the lesion stabilizer includes a suction tube configured to apply suction to the lesion and a suction pump configured to controllably deliver suction to the suction tube, and wherein the controller is configured to automatically cause the suction pump to deliver suction to the suction tube in response to and after the actuation of the user-operated control by the user, without any further command or action being taken by the user.

6. The automated skin biopsy apparatus of claim 5 wherein the suction tube has a suction pathway and includes a screen mounted within the suction pathway, and wherein the controller is configured to automatically cause the suction pump to stop delivering suction to the suction tube after the lesion has been resected from the skin.

7. The automated skin biopsy apparatus of claim 2 wherein the lesion stabilizer include a gripper configured to controllably grip the lesion and wherein the controller is configured to cause the gripper to grip the lesion in response to the actuation of the user-operated control by the user.

8. The automated skin biopsy apparatus of claim 2 wherein the lesion stabilizer is configured to controllably pull the lesion away from the skin and wherein the controller is configured to automatically cause the lesion stabilizer to pull the lesion away from the skin in response to and after the actuation of the user-operated control by the user, without any further command or action being taken by the user.

9. The automated skin biopsy apparatus of claim 1 further comprising an anesthesia applicator configured to controllably apply anesthesia in the area of the lesion and wherein the controller is configured to automatically cause the anesthesia applicator to apply anesthesia in the area of the lesion before the suture applicator sutures the skin in response to and after the actuation of the user-operated control by the user, without any further command or action being taken by the user.

10. The automated skin biopsy apparatus of claim 9 wherein the anesthesia applicator includes a plurality of closely-clustered needles having approximately the same length and configured to fed by a common supply of anesthesia.

11. The automated skin biopsy apparatus of claim 1 further comprising a camera configured to take a photograph of the lesion before the lesion is resected from the skin by the lesion resector.

12. The automated skin biopsy apparatus of claim 1 further comprising a printer configured to print a label for a specimen container that is configured to hold the resected lesion and wherein the controller is configured to cause the printer to print a label with information that identifies a patient associated with the lesion.

13. The automated skin biopsy apparatus of claim 1 wherein the lesion resector includes at least one cutting blade.

14. The automated skin biopsy apparatus of claim 1 wherein the suture applicator and the lesion resector are both part of a single tool.

15. An skin biopsy process for taking a biopsy of a lesion on skin comprising:
suturing skin surrounding the lesion; and
resecting the lesion from the skin after the suturing and while the sutures are in place.

16. The skin biopsy process of claim 15 further comprising initiating an automated process and wherein the suturing and the resecting are part of the automated process.

17. The skin biopsy process of claim 16 wherein the automated process includes stabilizing the lesion while on the skin before the suturing.

18. The skin biopsy process of claim 17 wherein the stabilizing includes applying suction to the lesion.

19. The skin biopsy process of claim 18 wherein the automated process further comprises pulling the lesion away from the skin before the resecting.

20. The skin biopsy process of claim 19 wherein the automated process further comprises holding the lesion in a holding area after the resecting.

21. The skin biopsy process of claim 17 wherein the stabilizing includes griping the lesion.

22. The skin biopsy process of claim 21 wherein the automated process further comprises pulling the lesion away from the skin before the resecting.

23. The skin biopsy process of claim 16 wherein the automated process includes applying anesthesia in the area of the lesion after the stabilizing and before the resecting.

24. The skin biopsy process of claim 23 wherein the applying anesthesia is performed by injecting anesthesia through a plurality of needles simultaneously.
